SEQUENCE LISTING (149 sequences)

SEQ ID NO: 1 - DNA, 30 bp, Artificial Sequence
  Description of Artificial Sequence Synthetic probe
tgtatgtggaagatgtagttacggatgcac

SEQ ID NO: 2 - DNA, 31 bp, Artificial Sequence
  Description of Artificial Sequence Synthetic probe
catgacgcatgtactctttataatcagaatt

SEQ ID NO: 3 - DNA, 30 bp, Artificial Sequence
  Description of Artificial Sequence Synthetic probe
tgtatgtagcagatttagacacagatgcac

SEQ ID NO: 4 - DNA, 30 bp, Artificial Sequence
  Description of Artificial Sequence Synthetic probe
catggcgcatgtattccttataatctgaat

SEQ ID NO: 5 - DNA, 30 bp, Artificial Sequence
  Description of Artificial Sequence Synthetic probe
gtagatatggcagcacataatgacatattt

SEQ ID NO: 6 - DNA, 30 bp, Artificial Sequence
  Description of Artificial Sequence Synthetic probe
ttctgaagtagatatggcagcacataatga

SEQ ID NO: 7 - DNA, 30 bp, Artificial Sequence
  Description of Artificial Sequence Synthetic probe
aactgtgcaaaataaccttaactgcagacg

SEQ ID NO: 8 - DNA, 31 bp, Artificial Sequence
  Description of Artificial Sequence Synthetic probe
atacatacattctatgaattccactattttg

SEQ ID NO: 9 - DNA, 30 bp, Artificial Sequence
  Description of Artificial Sequence Synthetic probe
cccaggaggcacactagaagatacttatag

SEQ ID NO: 10 - DNA, 30 bp, Artificial Sequence
  Description of Artificial Sequence Synthetic probe
atcatattgcccaggtacaggagactgtgt

SEQ ID NO: 11 - DNA, 29 bp, Artificial Sequence
  Description of Artificial Sequence Synthetic probe
cttattttcagccggtgcagcatcctttt

SEQ ID NO: 12 - DNA, 30 bp, Artificial Sequence
  Description of Artificial Sequence Synthetic probe
gaatagcagtattttagaggattggaactt

SEQ ID NO: 13 - DNA, 31 bp, Artificial Sequence
  Description of Artificial Sequence Synthetic probe
agttaaagttttggaatgtggatttaaagga

SEQ ID NO: 14 - DNA, 30 bp, Artificial Sequence
  Description of Artificial Sequence Synthetic probe
tggtttaaatggagtggatgcagatgctgc

SEQ ID NO: 15 - DNA, 30 bp, Artificial Sequence
  Description of Artificial Sequence Synthetic probe
atcttcctttggcacaggaggggcgttacg

SEQ ID NO: 16 - DNA, 30 bp, Artificial Sequence
  Description of Artificial Sequence Synthetic probe
aacaatttataagacatggcgaagaatatg

SEQ ID NO: 17 - DNA, 30 bp, Artificial Sequence
  Description of Artificial Sequence Synthetic probe
acatttaatgaatgcctccatattggagga

SEQ ID NO: 18 - DNA, 30 bp, Artificial Sequence
  Description of Artificial Sequence Synthetic probe
gtaacgcccctcctgtgccaaaggaagatc

SEQ ID NO: 19 - DNA, 30 bp, Artificial Sequence
  Description of Artificial Sequence Synthetic probe
cttgtggcagctgggggtgacaatccaata

SEQ ID NO: 20 - DNA, 30 bp, Artificial Sequence
  Description of Artificial Sequence Synthetic probe
gataacgtttgtgtggttgcagatatagtc

SEQ ID NO: 21 - DNA, 30 bp, Artificial Sequence
  Description of Artificial Sequence Synthetic probe
ttccagccctcaagtaaagtggagttcata

SEQ ID NO: 22 - DNA, 30 bp, Artificial Sequence
  Description of Artificial Sequence Synthetic probe
tgtagtatcactgtttgcaattgcagcaca

SEQ ID NO: 23 - DNA, 30 bp, Artificial Sequence
  Description of Artificial Sequence Synthetic probe
agaacctgagggaggtgtggtcaatccaaa

SEQ ID NO: 24 - DNA, 33 bp, Artificial Sequence
  Description of Artificial Sequence Synthetic probe
taaagagtatttaagacatggtgaggaatttga

SEQ ID NO: 25 - DNA, 31 bp, Artificial Sequence
  Description of Artificial Sequence Synthetic probe
catatattcacagtatgaatcctgctatttt

SEQ ID NO: 26 - DNA, 32 bp, Artificial Sequence
  Description of Artificial Sequence Synthetic probe
agttagtagacttgtatgtgtcttcagttgtt

SEQ ID NO: 27 - DNA, 30 bp, Artificial Sequence
  Description of Artificial Sequence Synthetic probe
ttcccaaaatgaatagtcagaaaaaggatc

SEQ ID NO: 28 - DNA, 30 bp, Artificial Sequence
  Description of Artificial Sequence Synthetic probe
tactgtcactagttacttgtgtgcataaag

SEQ ID NO: 29 - DNA, 30 bp, Artificial Sequence
  Description of Artificial Sequence Synthetic probe
gtatatttacctaaggggtcttccttttcc

SEQ ID NO: 30 - DNA, 30 bp, Artificial Sequence
  Description of Artificial Sequence Synthetic probe
aaaggaagaccccttaggtaaatatacatt

SEQ ID NO: 31 - DNA, 30 bp, Artificial Sequence
  Description of Artificial Sequence Synthetic probe
caactatgcaaagttaccttaactgcagaa

SEQ ID NO: 32 - DNA, 30 bp, Artificial Sequence
  Description of Artificial Sequence Synthetic probe
atatggtggagttgtacttgtggattgtgt

SEQ ID NO: 33 - DNA, 30 bp, Artificial Sequence
  Description of Artificial Sequence Synthetic probe
tccttaggaggttgcggacgctgacatgta

SEQ ID NO: 34 - DNA, 30 bp, Artificial Sequence
  Description of Artificial Sequence Synthetic probe
gtcactagaagacacagcagaacacacaga

SEQ ID NO: 35 - DNA, 30 bp, Artificial Sequence
  Description of Artificial Sequence Synthetic probe
aatggatcatctttaggttttggtgcactg

SEQ ID NO: 36 - DNA, 30 bp, Artificial Sequence
  Description of Artificial Sequence Synthetic probe
gtaccttagaggacacatatcgctatgtaa

SEQ ID NO: 37 - DNA, 30 bp, Artificial Sequence
  Description of Artificial Sequence Synthetic probe
atgtaacatcacaggctgtaacttgtcaaa

SEQ ID NO: 38 - DNA, 31 bp, Artificial Sequence
  Description of Artificial Sequence Synthetic probe
ggtatggaagactctatagaggtagataatg

SEQ ID NO: 39 - DNA, 30 bp, Artificial Sequence
  Description of Artificial Sequence Synthetic probe
gtatctgtaagtgtctaccaaactggcaga

SEQ ID NO: 40 - DNA, 30 bp, Artificial Sequence
  Description of Artificial Sequence Synthetic probe
gtagtaccaactttacattatctacctcta

SEQ ID NO: 41 - DNA, 30 bp, Artificial Sequence
  Description of Artificial Sequence Synthetic probe
ataycaggcacgtggaggagtatgatttac

SEQ ID NO: 42 - DNA, 30 bp, Artificial Sequence
  Description of Artificial Sequence Synthetic probe
aactgtgtactgtcacattaacaactgatg

SEQ ID NO: 43 - DNA, 30 bp, Artificial Sequence
  Description of Artificial Sequence Synthetic probe
aactgtgtactgtmacattaacaactgatg

SEQ ID NO: 44 - DNA, 30 bp, Artificial Sequence
  Description of Artificial Sequence Synthetic probe
cttgaaattactgttattatatggggttgg

SEQ ID NO: 45 - DNA, 30 bp, Artificial Sequence
  Description of Artificial Sequence Synthetic probe
cctccaacaacgtaggatccattgcatgaa

SEQ ID NO: 46 - DNA, 30 bp, Artificial Sequence
  Description of Artificial Sequence Synthetic probe
gtatatgtatcaccagatgttgcagtggca

SEQ ID NO: 47 - DNA, 30 bp, Artificial Sequence
  Description of Artificial Sequence Synthetic probe
tagcctgacagcgaatagcttctgattgta

SEQ ID NO: 48 - DNA, 30 bp, Artificial Sequence
  Description of Artificial Sequence Synthetic probe
gtatgtacaatcagaagctattcgctgtca

SEQ ID NO: 49 - DNA, 31 bp, Artificial Sequence
  Description of Artificial Sequence Synthetic probe
tacacaatatgaatcctaacatattagagga

SEQ ID NO: 50 - DNA, 30 bp, Artificial Sequence
  Description of Artificial Sequence Synthetic probe
gttgcaccaccaccttcaggaactttagaa

SEQ ID NO: 51 - DNA, 30 bp, Artificial Sequence
  Description of Artificial Sequence Synthetic probe
atatgtactgggcacagtagggtcagtaga

SEQ ID NO: 52 - DNA, 30 bp, Artificial Sequence
  Description of Artificial Sequence Synthetic probe
aagcagaggcaggtggggacacaccaaaat

SEQ ID NO: 53 - DNA, 30 bp, Artificial Sequence
  Description of Artificial Sequence Synthetic probe
tatatgtagacggaggggactgtgtagtgg

SEQ ID NO: 54 - DNA, 31 bp, Artificial Sequence
  Description of Artificial Sequence Synthetic probe
cgcatgtattgcttatattgttcactagtat

SEQ ID NO: 55 - DNA, 29 bp, Artificial Sequence
  Description of Artificial Sequence Synthetic probe
ctgcttttctggaggtgtagtatcctttt

SEQ ID NO: 56 - DNA, 30 bp, Artificial Sequence
  Description of Artificial Sequence Synthetic probe
ggcacaggattttgtgtagaggcacataat

SEQ ID NO: 57 - DNA, 30 bp, Artificial Sequence
  Description of Artificial Sequence Synthetic probe
atgaycctactaagtttaagcastatagta

SEQ ID NO: 58 - DNA, 30 bp, Artificial Sequence
  Description of Artificial Sequence Synthetic probe
tmcctccaccacctactacaagtttrgtgg

SEQ ID NO: 59 - DNA, 30 bp, Artificial Sequence
  Description of Artificial Sequence Synthetic probe
tcgttttgtgcaatcagttgctgttacctg

SEQ ID NO: 60 - DNA, 30 bp, Artificial Sequence
  Description of Artificial Sequence Synthetic probe
taaatgttggggaaaccgcagcagtggcag

SEQ ID NO: 61 - DNA, 30 bp, Artificial Sequence
  Description of Artificial Sequence Synthetic probe
tggaggggtgtccttttgacagctagtagc

SEQ ID NO: 62 - DNA, 30 bp, Artificial Sequence
  Description of Artificial Sequence Synthetic probe
ggcttatttacacacaatggatcctaccat

SEQ ID NO: 63 - DNA, 30 bp, Artificial Sequence
  Description of Artificial Sequence Synthetic probe
acaatggatcctaccattcttgaacagtgg

SEQ ID NO: 64 - DNA, 30 bp, Artificial Sequence
  Description of Artificial Sequence Synthetic probe
gattaacattacctccgtctgctagtttgg

SEQ ID NO: 65 - DNA, 30 bp, Artificial Sequence
  Description of Artificial Sequence Synthetic probe
ttatatgtgctttcctttttaacctcagca

SEQ ID NO: 66 - DNA, 29 bp, Artificial Sequence
  Description of Artificial Sequence Synthetic probe
gtgtcctccaaagatgcagacggtggtgg

SEQ ID NO: 67 - DNA, 30 bp, Artificial Sequence
  Description of Artificial Sequence Synthetic probe
aacctcagcagacagggatattttacatag

SEQ ID NO: 68 - DNA, 30 bp, Artificial Sequence
  Description of Artificial Sequence Synthetic probe
aagctagtggcaacaggaggcgacaaacct

SEQ ID NO: 69 - DNA, 30 bp, Artificial Sequence
  Description of Artificial Sequence Synthetic probe
gctatcctgcgtggatgctgtagcacacaa

SEQ ID NO: 70 - DNA, 30 bp, Artificial Sequence
  Description of Artificial Sequence Synthetic probe
aactacttgtagctgggggggttataccaa

SEQ ID NO: 71 - DNA, 30 bp, Artificial Sequence
  Description of Artificial Sequence Synthetic probe
atctgctgtaagggttatggtacataactg

SEQ ID NO: 72 - DNA, 30 bp, Artificial Sequence
  Description of Artificial Sequence Synthetic probe
tgtctaaggtactgattaatttttcgtgca

SEQ ID NO: 73 - DNA, 30 bp, Artificial Sequence
  Description of Artificial Sequence Synthetic probe
tttatcttctaggctggtggccactggcgg

SEQ ID NO: 74 - DNA, 30 bp, Artificial Sequence
  Description of Artificial Sequence Synthetic probe
tataattagtttctgtgkttacagtggcac

SEQ ID NO: 75 - DNA, 30 bp, Artificial Sequence
  Description of Artificial Sequence Synthetic probe
agtcctctagcaaccgcgcatccatgttat

SEQ ID NO: 76 - DNA, 30 bp, Artificial Sequence
  Description of Artificial Sequence Synthetic probe
atattcttcaacatgacgtacatattcctt

SEQ ID NO: 77 - DNA, 30 bp, Artificial Sequence
  Description of Artificial Sequence Synthetic probe
tcttctttagttacttcagtgcataatgtc

SEQ ID NO: 78 - DNA, 30 bp, Artificial Sequence
  Description of Artificial Sequence Synthetic probe
ccttccttagttacttcagtgcataatgtc

SEQ ID NO: 79 - DNA, 30 bp, Artificial Sequence
  Description of Artificial Sequence Synthetic probe
ctggcatattctttaaaactggtaggtgtg

SEQ ID NO: 80 - DNA, 30 bp, Artificial Sequence
  Description of Artificial Sequence Synthetic probe
tcctgtttaactggcggtgcggtgtccttt

SEQ ID NO: 81 - DNA, 30 bp, Artificial Sequence
  Description of Artificial Sequence Synthetic probe
tctttctgtgtgtgcttctactactbcttc

SEQ ID NO: 82 - DNA, 30 bp, Artificial Sequence
  Description of Artificial Sequence Synthetic probe
tttaaagaatatgccagacatgtggaggaa

SEQ ID NO: 83 - DNA, 30 bp, Artificial Sequence
  Description of Artificial Sequence Synthetic probe
aatgtcatacattcataatatgaataccac

SEQ ID NO: 84 - DNA, 30 bp, Artificial Sequence
  Description of Artificial Sequence Synthetic probe
tatattcagatacagggggggatgtagcag

SEQ ID NO: 85 - DNA, 30 bp, Artificial Sequence
  Description of Artificial Sequence Synthetic probe
ataacttggcatagcgatcctccttgggcg

SEQ ID NO: 86 - DNA, 30 bp, Artificial Sequence
  Description of Artificial Sequence Synthetic probe
atattccctaaagcttgtggctttatattc

SEQ ID NO: 87 - DNA, 30 bp, Artificial Sequence
  Description of Artificial Sequence Synthetic probe
gtggaagggggaggtaaaaccccaaagttc

SEQ ID NO: 88 - DNA, 30 bp, Artificial Sequence
  Description of Artificial Sequence Synthetic probe
atacgggtccaccttgggacgggtaggcag

SEQ ID NO: 89 - DNA, 30 bp, Artificial Sequence
  Description of Artificial Sequence Synthetic probe
aaatgtcatttgcgcatacgggtccacctt

SEQ ID NO: 90 - DNA, 30 bp, Artificial Sequence
  Description of Artificial Sequence Synthetic probe
gttaatgtgcttttagctgcattaatagtc

SEQ ID NO: 91 - DNA, 30 bp, Artificial Sequence
  Description of Artificial Sequence Synthetic probe
tggcgaaggtattgattgatttcacgkgca

SEQ ID NO: 92 - DNA, 30 bp, Artificial Sequence
  Description of Artificial Sequence Synthetic probe
cacatggcgaaggtattgattgatttcacg

SEQ ID NO: 93 - DNA, 30 bp, Artificial Sequence
  Description of Artificial Sequence Synthetic probe
taatactttattagacgattggaayattgg

SEQ ID NO: 94 - DNA, 30 bp, Artificial Sequence
  Description of Artificial Sequence Synthetic probe
aggataaatataggtatattaaaagcacag

SEQ ID NO: 95 - DNA, 30 bp, Artificial Sequence
  Description of Artificial Sequence Synthetic probe
tcttcctttgctgttggaggggatgttttt

SEQ ID NO: 96 - DNA, 30 bp, Artificial Sequence
  Description of Artificial Sequence Synthetic probe
tggtgtgtatgtattgcataacatttgcag

SEQ ID NO: 97 - DNA, 30 bp, Artificial Sequence
  Description of Artificial Sequence Synthetic probe
gttttcatttttgtatgtagcctctgattt

SEQ ID NO: 98 - DNA, 30 bp, Artificial Sequence
  Description of Artificial Sequence Synthetic probe
aggtgcaggggcgtctttttgacatgtaat

SEQ ID NO: 99 - DNA, 30 bp, Artificial Sequence
  Description of Artificial Sequence Synthetic probe
agcggtatgtatctacaagactagcagatg

SEQ ID NO: 100 - DNA, 31 bp, Artificial Sequence
  Description of Artificial Sequence Synthetic probe
gttgtgtactataacattgtcaactgatgta

SEQ ID NO: 101 - DNA, 31 bp, Artificial Sequence
  Description of Artificial Sequence Synthetic probe
gttgtgtactataacattatccactgatgta

SEQ ID NO: 102 - DNA, 30 bp, Artificial Sequence
  Description of Artificial Sequence Synthetic probe
gtgttgcccctccaccatctgctagtcttg

SEQ ID NO: 103 - DNA, 30 bp, Artificial Sequence
  Description of Artificial Sequence Synthetic probe
atggtttaaaagtggcagatgcagattgtg

SEQ ID NO: 104 - DNA, 30 bp, Artificial Sequence
  Description of Artificial Sequence Synthetic probe
tgtgcaggggcatcgcgttgacatgtagta

SEQ ID NO: 105 - DNA, 30 bp, Artificial Sequence
  Description of Artificial Sequence Synthetic probe
aaactttgtagggctatatacagcaggtat

SEQ ID NO: 106 - DNA, 30 bp, Artificial Sequence
  Description of Artificial Sequence Synthetic probe
tggtggaggggtaactcctatattccaatt

SEQ ID NO: 107 - DNA, 30 bp, Artificial Sequence
  Description of Artificial Sequence Synthetic probe
aatattccatgaaactagaggctttatatg

SEQ ID NO: 108 - DNA, 30 bp, Artificial Sequence
  Description of Artificial Sequence Synthetic probe
tttttctgcaggaggaggactgtttttctg

SEQ ID NO: 109 - DNA, 30 bp, Artificial Sequence
  Description of Artificial Sequence Synthetic probe
tctgatacagaggacgctgtggcagtacaa

SEQ ID NO: 110 - DNA, 30 bp, Artificial Sequence
  Description of Artificial Sequence Synthetic probe
gtggcgaagatactcacgaaaattagaagc

SEQ ID NO: 111 - DNA, 30 bp, Artificial Sequence
  Description of Artificial Sequence Synthetic probe
tagagttggcatacgttgtagtagagctac

SEQ ID NO: 112 - DNA, 30 bp, Artificial Sequence
  Description of Artificial Sequence Synthetic probe
gagttggcatacgttgtagtagagctacta

SEQ ID NO: 113 - DNA, 30 bp, Artificial Sequence
  Description of Artificial Sequence Synthetic probe
aggaggttgaggacgttggcaactaatagc

SEQ ID NO: 114 - DNA, 30 bp, Artificial Sequence
  Description of Artificial Sequence Synthetic probe
ctatgaattctactatattggaagagtgga

SEQ ID NO: 115 - DNA, 30 bp, Artificial Sequence
  Description of Artificial Sequence Synthetic probe
accccaccaccgtcaggtactttagaggaa

SEQ ID NO: 116 - DNA, 30 bp, Artificial Sequence
  Description of Artificial Sequence Synthetic probe
ttaaatttgcatagggattgggctttgctt

SEQ ID NO: 117 - DNA, 30 bp, Artificial Sequence
  Description of Artificial Sequence Synthetic probe
ttaaattggcatagggattaggctttgctt

SEQ ID NO: 118 - DNA, 30 bp, Artificial Sequence
  Description of Artificial Sequence Synthetic probe
agcagaaggcgattgtgaggtaggagcaca

SEQ ID NO: 119 - DNA, 30 bp, Artificial Sequence
  Description of Artificial Sequence Synthetic probe
agcaggaggggattgtgtagtaggcgcaca

SEQ ID NO: 120 - DNA, 30 bp, Artificial Sequence
  Description of Artificial Sequence Synthetic probe
ttctgcagcagcagatgtagctgtgcaaat

SEQ ID NO: 121 - DNA, 30 bp, Artificial Sequence
  Description of Artificial Sequence Synthetic probe
ctgtccaaaatgacatgtcggcataagggt

SEQ ID NO: 122 - DNA, 30 bp, Artificial Sequence
  Description of Artificial Sequence Synthetic probe
tgcaacagatggagtaacagcagtgctaat

SEQ ID NO: 123 - DNA, 30 bp, Artificial Sequence
  Description of Artificial Sequence Synthetic probe
tgcaactgatggagtagcagcagtgctaat

SEQ ID NO: 124 - DNA, 30 bp, Artificial Sequence
  Description of Artificial Sequence Synthetic probe
tgtagaatccatggtgtgcaggtaagccat

SEQ ID NO: 125 - DNA, 30 bp, Artificial Sequence
  Description of Artificial Sequence Synthetic probe
ttcattagcctgtgtagcagcagctgaaat

SEQ ID NO: 126 - DNA, 31 bp, Artificial Sequence
  Description of Artificial Sequence Synthetic probe
cactcatcyaataaatgttcattcatactat

SEQ ID NO: 127 - DNA, 30 bp, Artificial Sequence
  Description of Artificial Sequence Synthetic probe
atattctgattcggtgttggtagcagcact

SEQ ID NO: 128 - DNA, 30 bp, Artificial Sequence
  Description of Artificial Sequence Synthetic probe
aaataggacatgacctctggagtcagacgg

SEQ ID NO: 129 - DNA, 30 bp, Artificial Sequence
  Description of Artificial Sequence Synthetic probe
atatagatggaactggattagtagttgcag

SEQ ID NO: 130 - DNA, 30 bp, Artificial Sequence
  Description of Artificial Sequence Synthetic probe
cctttttttgtggaacaaccacatccttct

SEQ ID NO: 131 - DNA, 30 bp, Artificial Sequence
  Description of Artificial Sequence Synthetic probe
tccttaaagcgtgtagaactgtattctgtg

SEQ ID NO: 132 - DNA, 30 bp, Artificial Sequence
  Description of Artificial Sequence Synthetic probe
atctcaggcgttaggtgtatcttacatagt

SEQ ID NO: 133 - DNA, 30 bp, Artificial Sequence
  Description of Artificial Sequence Synthetic probe
aatggcccgagaggtaagaaagcgataggt

SEQ ID NO: 134 - DNA, 20 bp, Artificial Sequence
  Description of Artificial Sequence Synthetic primer
actaggatcatcgggaaaag

SEQ ID NO: 135 - DNA, 20 bp, Artificial Sequence
  Description of Artificial Sequence Synthetic probe
tggctctctattcaatcagc

SEQ ID NO: 136 - DNA, 30 bp, Artificial Sequence
  Description of Artificial Sequence Synthetic probe
ttctccacccactacgcacccccgccagca

SEQ ID NO: 137 - DNA, 37 bp, Artificial Sequence
  Description of Artificial Sequence Synthetic probe
gggctcaagctcctaatgccaaagacctactactctg

SEQ ID NO: 138 - DNA, 31 bp, Artificial Sequence
  Description of Artificial Sequence Synthetic probe
ctcattaggcaccccaggctttacactttat

SEQ ID NO: 139 - DNA, 35 bp, Artificial Sequence
  Description of Artificial Sequence Synthetic probe
tcactcattaggcaccccaggctttacactttatg

SEQ ID NO: 140 - DNA, 27 bp, Artificial Sequence
  Description of Artificial Sequence Synthetic probe
gcagtataagattattgatgccggaac

SEQ ID NO: 141 - DNA, 27 bp, Artificial Sequence
  Description of Artificial Sequence Synthetic probe
gtcaaaacctgggatagtagttttacc

SEQ ID NO: 142 - DNA, 20 bp, Artificial Sequence
  Description of Artificial Sequence Synthetic primer
cgtccmarrggawactgatc

SEQ ID NO: 143 - DNA, 20 bp, Artificial Sequence
  Description of Artificial Sequence Synthetic primer
gcmcagggwcataayaatgg

SEQ ID NO: 144 - DNA, 20 bp, Artificial Sequence
  Description of Artificial Sequence Synthetic primer
gcgacccaatgcaaattggt

SEQ ID NO: 145 - DNA, 30 bp, Artificial Sequence
  Description of Artificial Sequence Synthetic probe
caagctcctaatgccaaagacctactactc

SEQ ID NO: 146 - DNA, 35 bp, Artificial Sequence
  Description of Artificial Sequence Synthetic probe
gggctcaagctcctaatgccaaagacctactactc

SEQ ID NO: 147 - DNA, 35 bp, Artificial Sequence
  Description of Artificial Sequence Synthetic probe
gagtgagctgataccgctcgccgcagccgaacgac

SEQ ID NO: 148 - DNA, 30 bp, Artificial Sequence
  Description of Artificial Sequence Synthetic probe
gtagatatggcagcacatattgacatattt

SEQ ID NO: 149 - DNA, 30 bp, Artificial Sequence
  Description of Artificial Sequence Synthetic probe
gtagatatggcagctcataatgtcatattt

(19) United States
(12) Patent Application Publication
(10) Pub. No.: US 2011/0070576 A1
(43) Pub. Date: Mar. 24, 2011

(54) VITRO DIAGNOSTIC KIT FOR IDENTIFICATION OF HUMAN PAPILLOMAVIRUS IN CLINICAL SAMPLES

(75) Inventors: Maria Luisa Villahermosa jaen, San Javier (ES); Irene Gascon Escobar, Madrid (ES)

(73) Assignee: GENOMICA S.A.U., Madrid (ES)

(21) Appl. No.: 11/997,994

(22) PCT Filed: Aug. 4, 2006
(86) PCT No.: PCT/GB2006/050231

§ 371 Date: Sep. 2, 2008

(30) Foreign Application Priority Data
Aug. 5, 2005 (GB) 0516145.0

(51) Int. Cl. C12Q 1/68; C12M 1/34; C07H 21/00

(52) U.S. Cl. 435/6; 435/287.2; 536/24.32; 536/24.33

(57) ABSTRACT

A method and kit for detection and typing of HPV in a sample are described, as is a reaction vessel for use in the method. Universal HPV primers are used to amplify a sample by PCR; the amplified sample is then hybridised to an array of HPV type-specific probes to determine the HPV type.

### FIELD OF THE INVENTION

The present invention relates to an in vitro diagnostic kit and method for identification of Human Papillomavirus (HPV) in clinical samples. The invention also relates to apparatus for use in the kit and method.

More specifically, in preferred embodiments the present invention relates to an in vitro diagnostic kit for specific detection of human papillomavirus genotypes in clinical samples using probes for genotyping the HPV, a platform in which a nucleic acid array including the probes and a standard laboratory reaction vial are combined, a device for automatic processing of the results and a method for diagnosis of HPV infection using the in vitro diagnostic kit.

### BACKGROUND OF THE INVENTION

To date, around 100 Human Papillomavirus (HPV) types have been described. An HPV type is considered a new type when at least 10% of the gene sequences in the HPV regions E6, E7 and L1 differ from any previously known type. Subtypes, or variants, differ from the primary type by less than 2-5%. These viruses have tropism for human epithelia and have been linked to serious human diseases, especially carcinomas of the genital and oral mucosa.

About 50 HPV types have been isolated from the anogenital mucosa. They have been divided into low-risk types (e.g., HPV types 6, 11, 42, 43 and 44) and high-risk types (e.g., types 16, 18, 31, 33 and 45) depending on their association with cervical cancer. Detection and identification of HPV types is very important since persistent infection with high-risk types of HPVs is the main etiological factor for cervical cancer.

Detection and identification of HPV genotypes is carried out by HPV DNA testing. These methods can be done by direct detection of HPV DNA or by detection of amplified HPV DNA. Among methods for direct detection of HPV DNA are the Hybrid Capture (HC) method from Digene Corp., Gaithersburg, Md., USA and in situ hybridisation techniques. The HC is an FDA approved technique based on a signal-amplifying hybridization method. The hybridization probes which are used are HPV specific RNA sequences. After incubation of these probes with denatured HPV DNA from the clinical sample, RNA/DNA hybrids are formed that can be detected using a specific antibody. The HC method allows differentiation between high and low-risk HPV types, but it cannot identify the HPV type. An additional disadvantage of this test method is that the use of cocktail of probes frequently results in cross reactions between HPV types from the two classes.

Methods for identification of the HPV type via amplification of the viral genome are mainly carried out by polymerase chain reaction (PCR). Genotyping of HPV can be done by type-specific PCR using primers that recognize only one specific type. An alternative approach is the use of universal-primer PCR for amplification of all HPV types. The papillomaviruses are typed by subsequently analyzing the sequence of the amplified gene fragment. Analysis of this sequence can be performed by different methods, such as DNA sequencing, restriction fragment length polymorphism (RFLP) or nucleic acid hybridisation. Hybridisation techniques, such as reverse blot hybridisation, have been considered to be the most suitable for diagnostic purposes Meter et al. J Clin Microbiol. 1999, 37: 2508-2517; Van den Brule et al. J. Clin Microbiol. 2002, 40: 779-787).

Recently, microarray technology has been developed (see for example U.S. Pat. No. 5,445,934). The term microarray is meant to indicate analysis of many small spots to facilitate large scale nucleic acid analysis enabling the simultaneous analysis of thousands of DNA sequences. As is known in the art, reverse blotting is usually performed on membranes, whereas microarray is usually performed on a solid support and may also be performed on smaller scale. The microarray technology has been successfully applied to the field of HPV diagnosis (see Patent Publications WO0168915 and No. CA2484681).

However, there is still a drawback with the use of microarray technology that expensive equipment and laborious handling are required. This inconvenience is addressed by Patent Application No. US2005064469 where an ‘array-tube’ is provided. The term ‘array-tube’ describes a reaction vessel which has a shape and size typical of a laboratory reaction vessel (for example, a 1.5 ml Eppendorf tube) with a microarray arranged on its base in which microarray based tests can be carried out.

### AIMS OF THE INVENTION

In view of the above, it is an aim of the present invention to provide a reliable method for specific identification of HPV types possibly present in a clinical sample.

It is more particularly an aim of the present invention to provide a method for specific identification of HPV types using the ‘array-tube’ platform.

It is also an aim of the present invention to provide probes for specific detection and/or identification of different HPV types.

It is furthermore an aim of the present invention to provide a kit for detection and/or identification of HPV types comprising reagents, protocols and HPV specific probes arranged on an ‘array-tube’, allowing the reliable specific detection and/or identification of HPV types possibly present in a clinical sample.

### SUMMARY OF THE INVENTION

According to a first aspect of the invention, there is provided an assay for detecting and typing human papillomavirus (HPV) in a sample, the assay comprising: performing a nucleic acid amplification reaction on a sample, the amplification reaction being intended to amplify an HPV target sequence in a non-type specific manner; obtaining single stranded oligonucleotides from any amplification products; allowing single stranded oligonucleotides to hybridise where possible with the a plurality of HPV type-specific probes provided on a solid support, the support being located within a reaction vessel suitable for containing the sample; and detecting hybridised oligonucleotides.

Aspects of the invention also provide an assay for detecting and typing human papillomavirus virus (HPV) in a sample, the assay comprising: performing a nucleic acid amplification reaction on a sample, the sample being in contact with a solid support having a plurality of HPV type-specific probes immobilised thereon, the amplification reaction being intended to amplify an HPV target sequence in a non-type specific manner; obtaining single stranded oligonucleotides from any amplification products; allowing single stranded oligonucleotides to hybridise where possible with the HPV type-specific probes; and detecting hybridised oligonucleotides.

The amplification reaction is preferably PCR. Single stranded oligonucleotides may be obtained by denaturing any double stranded oligonucleotides present, for example by heating. Single stranded oligonucleotides are preferably allowed to hybridise under stringent conditions; such conditions will be understood to those of skill in the art, but preferably include incubating denatured oligonucleotides at 55° C. with the target, in a buffer comprising 1×SSC.

In preferred embodiments, the sample and the solid support are contained within a reaction vessel; for example, that described in US2005064469.

Preferably probes specific for at least 5, 10, 15, 20, 25, 30, 35, 40, or 42 HPV types are used, which are preferably selected from HPV types 6, 11, 16, 18, 26, 30, 31, 32, 33, 34/64, 35, 39, 40, 42, 43, 44, 45, 51, 52, 53, 54, 56, 57, 58, 59, 61, 62, 66, 67, 68, 69, 70, 71, 72, 73, 74, 81, 82, 83, 84, 85 and 89. The probes are conveniently 20 to 40 nt in length, preferably 25 to 35 nt, more preferably 28 to 32 nt, and most preferably around 30 nt. All probes need not be the same length. The probes are conveniently specific to the L1 region of HPV. Each type-specific probe may differ from probes specific to another HPV type in at least 1, 2, 3, or preferably more than 3 nt. Preferred probes are selected from the group comprising SEQ ID NO 1 to SEQ ID NO 133; several of these probes detect the same HPV type as described below. Preferably a plurality of probes are specific for the same HPV type, and more preferably at least two probes specific for each HPV type to be detected are used. Mixtures of these probes may be immobilised to the same location on the solid support, or each type-specific probe may be immobilised in a different location. Each probe specific for the same HPV type preferably detects a different portion of the HPV target sequence.

The probes may be duplicated on the solid support, to provide for multiple detection locations for redundancy.

One or more control sequences may also be detected; for example, a probe immobilised to the solid support which does not hybridise to the target sequence from any HPV type. The probe may be for a human genomic target sequence; the assay may then comprise amplifying the human target sequence from the sample and detecting whether amplification has occurred. A further control may be introduced by using non-specific labelled sequences immobilised to the solid support; detection of the label can ensure that the label is working properly. A still further control may be provided by including a control amplification sequence which may be amplified by the same primers as the human target, but which will be detected by a different oligonucleotide on the solid support. This control ensures that amplification is working correctly.

The invention also provides a reaction vessel including a solid support having a plurality of HPV type-specific probes immobilised thereon. Also provided is a kit for the detection and typing of HPV comprising such a reaction vessel, in combination with a nucleic acid amplification mix. The mix may comprise HPV consensus primers such as MY09 and MY11; and optionally HMB01; primers for amplifying a human target sequence; and a control amplification target sequence including sequences corresponding to flanking portions of the human target sequence, such that amplification of both target sequences will occur using the same primers. The kit may also include instructions for its use.

### BRIEF DESCRIPTION OF THE DRAWINGS

FIG. 1 shows an arrangement of probes on the surface of a microarray with 12×11=132 locations. Numbers correspond to the SEQ ID NO from the sequence listing. Single probes were fixed at two different locations for detection of 21 different HPV types, DNA sample quality control, and amplification control. LR=probes for location reference (SEQ ID NO 140+SEQ ID NO 141).

FIG. 2 shows an arrangement of probes on the surface of a microarray with 12×11=132 locations. Numbers correspond to the SEQ ID NO from the sequence listing. Single probes or mixtures of probes were fixed at two different locations for detection of 23 different HPV types, DNA sample quality control, and amplification control. LR=probes for location reference (SEQ ID NO 140+SEQ ID NO 141).

FIG. 3 shows an arrangement of probes on the surface of a microarray with 12×11=132 locations. Numbers correspond to the SEQ ID NO from the sequence listing. Mixtures of probes were fixed at two different locations for detection of 42 different HPV types and DNA sample quality control. LR=probes for location reference (SEQ ID NO 140+SEQ ID NO 141); M1=SEQ ID NO 76+SEQ ID NO 77+SEQ ID NO 78; M2=SEQ ID NO 122+SEQ ID NO 123+SEQ ID NO 124; M3=SEQ ID NO 116+SEQ ID NO 117+SEQ ID NO 118+SEQ ID NO 119.

FIG. 4 shows an arrangement of probes on the surface of a microarray with 12×10=120 locations. Numbers correspond to the SEQ ID NO from the sequence listing. Single probe or mixtures of probes were fixed at three different locations for detection of 35 different HPV types, DNA sample quality control, and amplification control. LR=probes for location reference (SEQ ID NO 140+SEQ ID NO 141); M1=SEQ ID NO 76+SEQ ID NO 77+SEQ ID NO 78; M2=SEQ ID NO 122+SEQ ID NO 123+SEQ ID NO 124.

FIG. 5 shows an arrangement of probes on the surface of a microarray with 12×10=120 locations. Numbers correspond to the SEQ ID NO from the sequence listing. Single probes or mixtures of probes were fixed at two different locations for detection of 14 different HPV types, DNA sample quality control, and amplification control. LR=probes for location reference (SEQ ID NO 140+SEQ ID NO 141); M4=SEQ ID NO 100+SEQ ID NO 101+SEQ ID NO 102.

FIG. 6 shows a schematic representation of recombinant plasmid pPG44 used in the PCR reaction as amplification positive control.

FIG. 7 shows a photograph of an ‘array tube’ used in the present invention.

### DETAILED DESCRIPTION OF THE INVENTION

The method for specific detection and/or identification of HPV types comprises following steps:

(i) Amplification of sample DNA: DNA obtained from clinical samples is amplified, preferably by PCR, using universal primers for all HPV known types which flank a genome region variable enough to allow further genotyping. Although the PCR is the preferred amplification method, amplification of target sequences in a sample may be accomplished by any other method known in the art (ligase chain reaction, transcription-based amplification system, strand displacement amplification, etc). In an embodiment of the present invention, primers MY11 and MY09 have been used (Manos et al., Molecular Diagnostics of Human Cancer; Furth M, Greaves M F, eds.; Cold Spring Harbor Press. 1989, vol. 7: 209-214), which amplify the variable L1 region.

A label is introduced in the amplified DNA during its amplification to allow further detection, preferably a label that provide a signal that may be detected by colorimetric methods. In a preferred embodiment, at least one of the primers used is labelled at the 5′ end with biotin. However, any other kind of label known in the art may be used (e. g. digoxigenin). Furthermore, labelling of amplified DNA may be alternatively achieved by adding modified nucleotides bearing a label (e. g. biotinylated or digoxigenin dUTP derivatives) in the PCR mixture. Radioactive labels may be used, or fluorophores, in certain embodiments.

(ii) Hybridization: amplified DNA from step (i) is denatured (e.g. by heat) and applied to an ‘array-tube’ with one or more probes from those shown in Table 1 (SEQ ID NO: 1-133). Other ways to prepare single stranded DNA after amplification may be used as well. Each probe shown in Table 1 (SEQ ID NO: 1-133) is capable of specific hybridization with the amplified L1 region from step (i) of only one HPV type, and thus enables specific identification of this HPV type, when this type is present in a biological sample. The different types of HPV in a sample can be identified by hybridization of amplified DNA from said types of HPV to at least one, but preferably more than one probe.

(iii) Detection: DNA hybrids may be detected by recognition of the label by specific binding to a ligand or by immunodetection. In the preferred embodiment, biotin label is detected by specific binding to streptavidin conjugated with horse-radish-peroxidase (HRP) and the subsequent conversion of tetramethylbenzidine (TMB) to a blue pigment that precipitates in the concrete location where corresponding specific probe was bound. Other kind of conjugates well known in the art may also be suitable for purposes of the present invention (e. g. streptavidin-Au conjugate). Fluorescently labelled detection systems may instead be used, either indirectly or directly labelled. Alternatively, other enzyme-based systems may be used.

(iv) Analysis and processing of the results: ‘array-tubes’ so processed can be read using simple optical devices, such as an optical microscope or ATR01 and ATS readers manufactured by CLONDIAG chip technologies GmbH (Jena, Germany)

In an alternative embodiment, the amplification and hybridisation steps may be performed in the same array-tube; that is, a sample is added to the array-tube, which sample is then amplified and hybridised to probes within the tube.

One process for preparing the ‘array-tube’ is disclosed in Patent Application No. US2005064469. In a preferred embodiment of the present invention, 5′ amine-linked oligonucleotide probes are bound to the surface of a solid support in known distinct locations. Said probes may be immobilized individually or as mixtures to delineated locations on the solid support. In a preferred embodiment, two type specific probes are used for each HPV type, which provides additional assurance that all HPV will be typed correctly including variants where nucleotide changes in the region of one type specific probe have occurred. Preferably two type-specific probes are employed that are capable of hybridizing in separate regions of the amplified product.

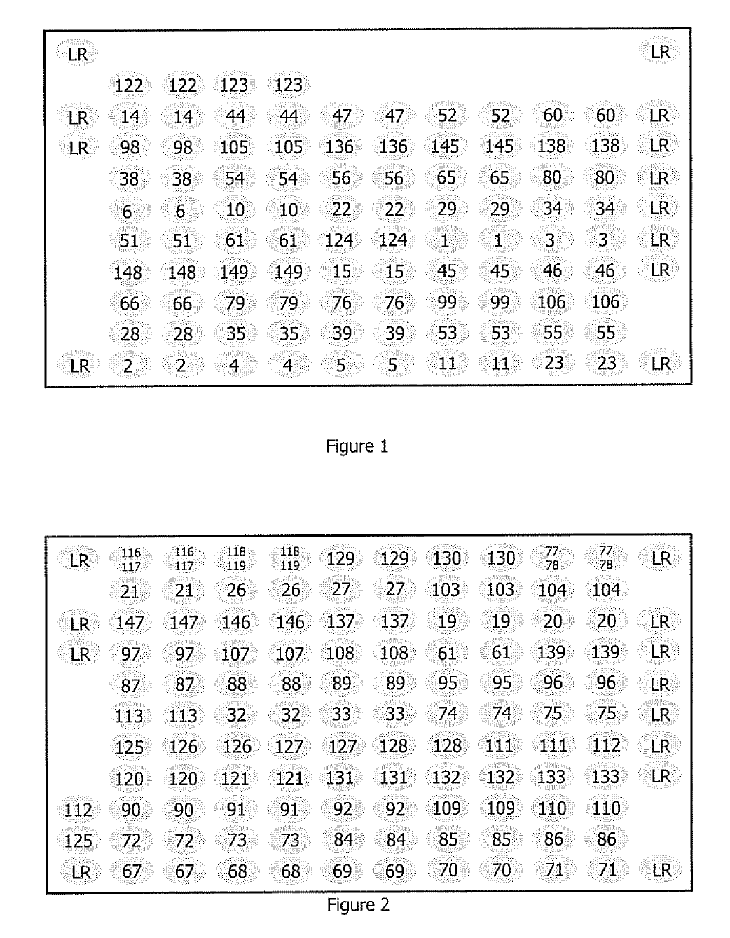

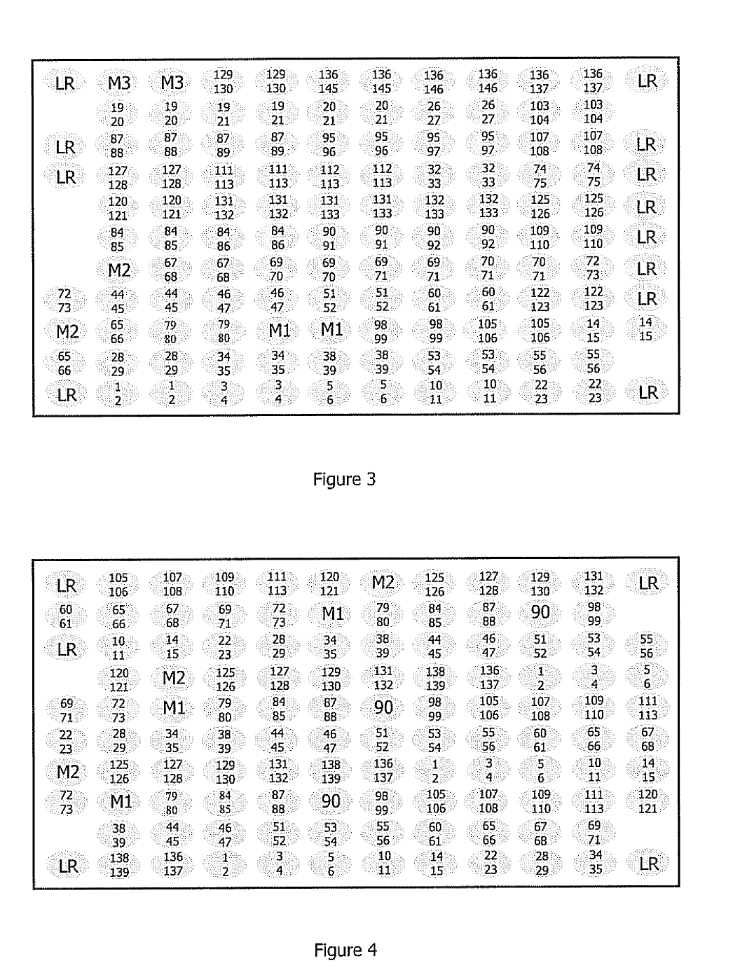

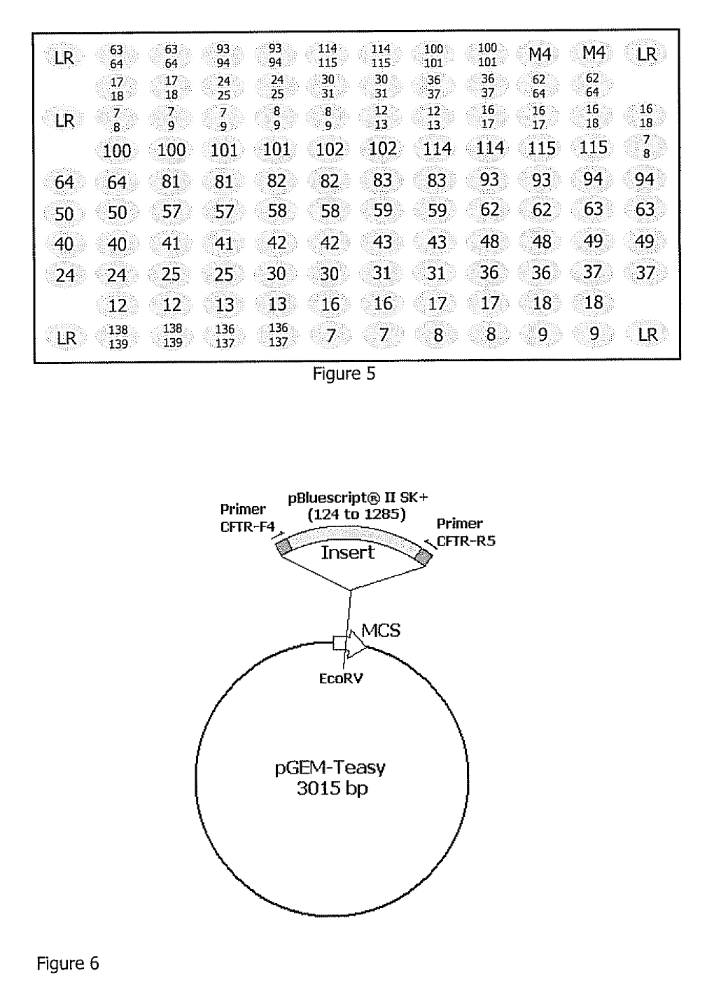

Said probes or mixtures of probes may be immobilized in a single location of the solid support, preferably in two distinct locations of the solid support and more preferably in three distinct locations of the solid support. FIGS. 1 to 5 exemplify schematic representations for different arrangements of probes on the surface of the microarray.

The ‘array-tube’ used in the present invention may comprise one or more HPV probes selected from nucleotide sequences from the sequence list (SEQ ID NO: 1-133). In addition, it may comprise one or more probes for specific detection of controls such as PCR reaction control or adequacy of the DNA from the sample control. Furthermore, it may also comprise one or more labelled oligonucleotides (e.g. biotin modified oligonucleotides) for positive control of the detection reaction and for positioning reference so that all remaining probes can be located.

Specific probes for HPV type identification were designed as follows. Sequences for all reference HPVs deposited in GenBank, including known variants, for the amplified L1 region were aligned using a conventional nucleic acid alignment program, such as BioEdit (4.8.6. version; Hall. Nucl Acids Symp Ser. 1999, 41:95-98) and most variable sequences regions among different HPV types were located. Potential sequences of oligonucleotides to be used as specific probes were selected from these variable sequences regions, preferably having following features: length of 20 to 40 bases, preferably an approximate length of 30 bases; preferably with no secondary structures or strings of consecutive same nucleotide longer than 4; preferably with a G+C ratio of 50% and a Tm as much similar among all selected probes as possible; and preferably with the mismatched nucleotides among the different HPV types sequences as much in the centre of the oligonucleotide sequence as possible.

Each potential probe sequence selected as aforementioned was compared against all known HPV sequences in the amplified L1 region using the BLAST program form the NCBI webpage (Altschul et al. Nucleic Acid Res. 1997, 25: 3389-3402). Finally, probes having at least three nucleotide mismatches when compared with all known HPV types (except when compared to the HPV type that the oligonucleotide probe is specific for) were chosen, with a preference for probes with greater than three mismatches.

The present invention provides probes for specific detection of the 42 most clinically important HPV types: 6, 11, 16, 18, 26, 30, 31, 32, 33, 34/64, 35, 39, 40, 42, 43, 44, 45, 51, 52, 53, 54, 56, 57, 58, 59, 61, 62, 66, 67, 68, 69, 70, 71, 72, 73, 74, 81, 82, 83, 84, 85 and 89 (Table 1; SEQ ID NO 1-133). Probes sequences are represented as single stranded DNA oligonucleotides from the 5′ to the 3′ end. In a preferred embodiment of the present invention, probes sequences correspond to the antisense strand, but it is obvious to anyone skilled in the art that any of these probes can be used as such, or in their complementary form, or in their RNA form (wherein T is replaced by U). The probes of the present invention can also be prepared by adding or changing one or more nucleotides of their sequence without dramatically affecting its functionality.

TABLE 1

| SEQ | | | |
|---|---|---|---|
| ID | Probe | HPV | |
| NO | name | type | Sequence (5′ -> 3′) |
| 1 | 6A1-AS | 6 | TGTATGTGGAAGATGTAGTTACGGATGCAC |
| 2 | 6134 | 6 | CATGACGCATGTACTCTTTATAATCAGAATT |
| 3 | 11A1-AS | 11 | TGTATGTAGCAGATTTAGACACAGATGCAC |
| 4 | 1131 | 11 | CATGGCGCATGTATTCCTTATAATCTGAAT |
| 5 | 16A | 16 | GTAGATATGGCAGCACATAATGACATATTT |
| 6 | 16E-AS | 16 | TTCTGAAGTAGATATGGCAGCACATAATGA |
| 7 | 16C3 | 16 | CGTCTGCAGTTAAGGTTATTTTGCACAGTT |
| 8 | 16C4 | 16 | CAAAATAGTGGAATTCATAGAATGTATGTAT |
| 9 | 16C5 | 16 | CTATAAGTATCTTCTAGTGTGCCTCCTGGG |
| 10 | 18A1-AS | 18 | ATCATATTGCCCAGGTACAGGAGACTGTGT |
| 11 | 18B3 | 18 | CTTATTTTCAGCCGGTGCAGCATCCTTTT |
| 12 | 18C2 | 18 | AAGTTCCAATCCTCTAAAATACTGCTATTC |
| 13 | 18C3 | 18 | TCCTTTAAATCCACATTCCAAAACTTTAACT |
| 14 | 26A1-AS | 26 | TGGTTTAAATGGAGTGGATGCAGATGCTGC |
| 15 | 2632 | 26 | ATCTTCCTTTGGCACAGGAGGGGCGTTACG |
| 16 | 26C1 | 26 | CATATTCTTCGCCATGTCTTATAAATTGTT |
| 17 | 26C3 | 26 | TCCTCCAATATGGAGGCATTCATTAAATGT |
| 18 | 26C4 | 26 | GATCTTCCTTTGGCACAGGAGGGGCGTTAC |
| 19 | 30A1 | 30 | CTTGTGGCAGCTGGGGGTGACAATCCAATA |
| 20 | 30B1 | 30 | GATAACGTTTGTGTGGTTGCAGATATAGTC |
| 21 | 30C1 | 30 | TTCCAGCCCTCAAGTAAAGTGGAGTTCATA |
| 22 | 31A-AS | 31 | TGTAGTATCACTGTTTGCAATTGCAGCACA |
| 23 | 31B5 | 31 | AGAACCTGAGGGAGGTGTGGTCAATCCAAA |
| 24 | 31C2 | 31 | TCAAATTCCTCACCATGTCTTAAATACTCTT |
| | | | TA |
| 25 | 31C5 | 31 | AAAATAGCAGGATTCATACTGTGAATATATG |
| 26 | 32A1 | 32 | AGTTAGTAGACTTGTATGTGTCTTCAGTTGT |
| | | | T |
| 27 | 32B1 | 32 | TTCCCAAAATGAATAGTCAGAAAAAGGATC |
| 28 | 33A2 | 33 | TACTGTCACTAGTTACTTGTGTGCATAAAG |
| 29 | 33B1-AS | 33 | GTATATTTACCTAAGGGGTCTTCCTTTTCC |
| 30 | 33C1 | 33 | AATGTATATTTACCTAAGGGGTCTTCCTTT |
| 31 | 33C3 | 33 | TTCTGCAGTTAAGGTAACTTTGCATAGTTG |
| 32 | 34/64A1 | 34/64 | ATATGGTGGAGTTGTACTTGTGGATTGTGT |
| 33 | 34/64B1 | 34/64 | TCCTTAGGAGGTTGCGGACGCTGACATGTA |
| 34 | 35A1-AS | 35 | GTCACTAGAAGACACAGCAGAACACACAGA |
| 35 | 35B1 | 35 | AATGGATCATCTTTAGGTTTTGGTGCACTG |
| 36 | 35C4 | 35 | TTACATAGCGATATGTGTCCTCTAAGGTAC |
| 37 | 35C7 | 35 | TTTGACAAGTTACAGCCTGTGATGTTACAT |
| 38 | 39A1-AS | 39 | GGTATGGAAGACTCTATAGAGGTAGATAATG |
| 39 | 39B1 | 39 | GTATCTGTAAGTGTCTACCAAACTGGCAGA |
| 40 | 39C2 | 39 | TAGAGGTAGATAATGTAAAGTTGGTACTAC |
| 41 | 39C3d | 39 | GTAAATCATACTCCTCCACGTGCCTGRTAT |
| 42 | 39C4 | 39 | CATCAGTTGTTAATGTGACAGTACACAGTT |
| 43 | 39C4d | 39 | CATCAGTTGTTAATGTGACAGTACACAGTT |
| 44 | 40A1-AS | 40 | CT TGAAATTACTGTTATTATATGGGGTTGG |
| 45 | 40B1 | 40 | CCTCCAACAACGTAGGATCCATTGCATGAA |
| 46 | 42A1 | 42 | GTATATGTATCACCAGATGTTGCAGTGGCA |
| 47 | 42B1-AS | 42 | TAGCCTGACAGCGAATAGCTTCTGATTGTA |
| 48 | 42C1 | 42 | TGACAGCGAATAGCTTCTGATTGTACATAC |
| 49 | 42C5 | 42 | TCCTCTAATATGTTAGGATTCATATTGTGTA |
| 50 | 42C6 | 42 | TTCTAAAGTTCCTGAAGGTGGTGGTGCAAC |
| 51 | 43A1 | 43 | ATATGTACTGGGCACAGTAGGGTCAGTAGA |
| 52 | 43B1-AS | 43 | AAGCAGAGGCAGGTGGGGACACACCAAAAT |
| 53 | 44A1 | 44 | TATATGTAGACGGAGGGGACTGTGTAGTGG |
| 54 | 44B1-AS | 44 | CGCATGTATTGCTTATATTGTTCACTAGTAT |
| 55 | 45B1 | 45 | CTGCTTTTCTGGAGGTGTAGTATCCTTTT |
| 56 | 45B4-AS | 45 | GGCACAGGATTTTGTGTAGAGGCACATAAT |
| 57 | 45C1d | 45 | TACTATASTGCTTAAACTTAGTAGGRTCAT |
| 58 | 45C3d | 45 | CCACYAAACTTGTAGTAGGTGGTGGAGGKA |
| 59 | 45C4 | 45 | CAGGTAACAGCAACTGATTGCACAAAACGA |
| 60 | 51A1-AS | 51 | TAAATGTTGGGGAAACCGCAGCAGTGGCAG |
| 61 | 51B1 | 51 | TGGAGGGGTGTCCTTTTGACAGCTAGTAGC |
| 62 | 51C3 | 51 | ATGGTAGGATCCATTGTGTGTAAATAAGCC |
| 63 | 51C4 | 51 | CCACTGTTCAAGAATGGTAGGATCCATTGT |
| 64 | 51C5 | 51 | CCAAACTAGCAGACGGAGGTAATGTTAATC |
| 65 | 52A1-AS | 52 | TTATATGTGCTTTCCTTTTTAACCTCAGCA |
| 66 | 52B2 | 52 | GTGTCCTCCAAAGATGCAGACGGTGGTGG |
| 67 | 53A1 | 53 | AACCTCAGCAGACAGGGATATTTTACATAG |
| 68 | 53B1 | 53 | AAGCTAGTGGCAACAGGAGGCGACAAACCT |
| 69 | 54A1 | 54 | GCTATCCTGCGTGGATGCTGTAGCACACAA |
| 70 | 54B1 | 54 | AACTACTTGTAGCTGGGGGGGTTATACCAA |
| 71 | 54C1 | 54 | ATCTGCTGTAAGGGTTATGGTACATAACTG |
| 72 | 56A1 | 56 | TGTCTAAGGTACTGATTAATTTTTCGTGCA |
| 73 | 56B1 | 56 | TTTATCTTCTAGGCTGGTGGCCACTGGCGG |
| 74 | 57A1d | 57 | TATAATTAGTTTCTGTGKTTACAGTGGCAC |
| 75 | 57B1 | 57 | AGTCCTCTAGCAACCGCGCATCCATGTTAT |
| 76 | 58A1 | 58 | ATATTCTTCAACATGACGTACATATTCCTT |
| 77 | 58B1a | 58 | TCTTCTTTAGTTACTTCAGTGCATAATGTC |
| 78 | 58B1b | 58 | CCTTCCTTAGTTACTTCAGTGCATAATGTC |
| 79 | 59A2 | 59 | CTGGCATATTCTTTAAAACTGGTAGGTGTG |
| 80 | 59B1-AS | 59 | TCCTGTTTAACTGGCGGTGCGGTGTCCTTT |
| 81 | 59C3_3d | 59 | GAAGVAGTAGTAGAAGCACACACAGAAAGA |
| 82 | 59C4 | 59 | TTCCTCCACATGTCTGGCATATTCTTTAAA |
| 83 | 59C6 | 59 | GTGGTATTCATATTATGAATGTATGACATT |
| 84 | 61A1 | 61 | TATATTCAGATACAGGGGGGGATGTAGCAG |
| 85 | 61B1 | 61 | ATAACTTGGCATAGCGATCCTCCTTGGGCG |
| 86 | 61B2 | 61 | ATATTCCCTAAAGCTTGTGGCTTTATATTC |
| 87 | 62A1 | 62 | GTGGAAGGGGGAGGTAAAACCCCAAAGTTC |
| 88 | 62B1 | 62 | ATACGGGTCCACCTTGGGACGGGTAGGCAG |
| 89 | 62B2 | 62 | AAATGTCATTTGCGCATACGGGTCCACCTT |
| 90 | 66A1 | 66 | GTTAATGTGCTTTTAGCTGCATTAATAGTC |
| 91 | 66B1 | 66 | TGGCGAAGGTATTGATTGATTTCACGKGCA |
| 92 | 66B2 | 66 | CACATGGCGAAGGTATTGATTGATTTCACG |
| 93 | 66C3d | 66 | CCAATRTTCCAATCGTCTAATAAAGTATTA |
| 94 | 66C4 | 66 | CTGTGCTTTTAATATACCTATATTTATCCT |
| 95 | 67A1 | 67 | TCTTCCTTTGCTGTTGGAGGGGATGTTTTT |
| 96 | 67B1 | 67 | TGGTGTGTATGTATTGCATAACATTTGCAG |
| 97 | 67B2 | 67 | GTTTTCATTTTTGTATGTAGCCTCTGATTT |
| 98 | 68A1-AS | 68 | AGGTGCAGGGGCGTCTTTTTGACATGTAAT |
| 99 | 68B1 | 68 | AGCGGTATGTATCTACAAGACTAGCAGATG |
| 100 | 68C4b | 68 | TACATCAGTTGACAATGTTATAGTACACAAC |
| 101 | 68C4c | 68 | TACATCAGTGGATAATGTTATAGTACACAAC |
| 102 | 68C7 | 68 | CAAGACTAGCAGATGGTGGAGGGGCAACAC |
| 103 | 69A1 | 69 | ATGGTTTAAAAGTGGCAGATGCAGATTGTG |
| 104 | 69B1 | 69 | TGTGCAGGGGCATCGCGTTGACATGTAGTA |
| 105 | 70A1-AS | 70 | AAACTTTGTAGGGCTATATACAGCAGGTAT |
| 106 | 70B1 | 70 | TGGTGGAGGGGTAACTCCTATATTCCAATT |
| 107 | 71A1 | 71 | AATATTCCATGAAACTAGAGGCTTTATATG |
| 108 | 71B1 | 71 | TTTTTCTGCAGGAGGAGGACTGTTTTTCTG |
| 109 | 72A1 | 72 | TCTGATACAGAGGACGCTGTGGCAGTACAA |
| 110 | 72B1 | 72 | GTGGCGAAGATACTCACGAAAATTAGAAGC |
| 111 | 73A1 | 73 | TAGAGTTGGCATACGTTGTAGTAGAGCTAC |
| 112 | 73A2 | 73 | GAGTTGGCATACGTTGTAGTAGAGCTACTA |
| 113 | 73B1 | 73 | AGGAGGTTGAGGACGTTGGCAACTAATAGC |
| 114 | 73C3 | 73 | TCCACTCTTCCAATATAGTAGAATTCATAG |
| 115 | 73C4 | 73 | TTCCTCTAAAGTACCTGACGGTGGTGGGGT |
| 116 | 74A1a | 74 | TTAAATTTGCATAGGGATTGGGCTTTGCTT |
| 117 | 74A1b | 74 | TTAAATTGGCATAGGGATTAGGCTTTGCTT |
| 118 | 74B1a | 74 | AGCAGAAGGCGATTGTGAGGTAGGAGCACA |
| 119 | 74B1b | 74 | AGCAGGAGGGGATTGTGTAGTAGGCGCACA |
| 120 | 81A1 | 81 | TTCTGCAGCAGCAGATGTAGCTGTGCAAAT |
| 121 | 81B1 | 81 | CTGTCCAAAATGACATGTCGGCATAAGGGT |
| 122 | 82A2a-AS | 82 | TGCAACAGATGGAGTAACAGCAGTGCTAAT |
| 123 | 82A2b-AS | 82 | TGCAACTGATGGAGTAGCAGCAGTGCTAAT |
| 124 | 82B1 | 82 | TGTAGAATCCATGGTGTGCAGGTAAGCCAT |
| 125 | 83A1 | 83 | TTCATTAGCCTGTGTAGCAGCAGCTGAAAT |
| 126 | 83B1d | 83 | CACTCATCYAATAAATGTTCATTCATACTAT |
| 127 | 84A1 | 84 | ATATTCTGATTCGGTGTTGGTAGCAGCACT |
| 128 | 84B1 | 84 | AAATAGGACATGACCTCTGGAGTCAGACGG |
| 129 | 85A1 | 85 | ATATAGATGGAACTGGATTAGTAGTTGCAG |
| 130 | 85B1 | 85 | CCTTTTTTTGTGGAACAACCACATCCTTCT |
| 131 | 89A1 | 89 | TCCTTAAAGCGTGTAGAACTGTATTCTGTG |
| 132 | 89B1 | 89 | ATCTCAGGCGTTAGGTGTATCTTACATAGT |
| 133 | 89C1 | 89 | AATGGCCCGAGAGGTAAGAAAGCGATAGGT |

Nucleotides of the sequences are designated as follows: G for Guanine, A for Adenine, T for Thymine, C for Cytosine, R for G or A, Y for T or C, M for A or C, K for G or T, S for G or C, W for A or T, H for A or C or T, B for G or T or C, V for G or C or A, D for G or A or T, and finally, N for G or A or T or C. The nucleotides as used in the present invention may be ribonucleotides, deoxyribonucleotides and modified nucleotides such as inosine or nucleotides containing modified groups which do not essentially after their hybridization characteristics.

The probes of the present invention can be obtained by different methods, such as chemical synthesis (e. g. by the conventional phosphotriester method) or genetic engineering techniques, for example by molecular cloning of recombinant plasmids in which corresponding nucleotide sequences have been inserted and can be latter obtained by digestion with nucleases.

For some HPV types, probes were designed from a sequence region that contained distinct nucleotides at a concrete position for different variants of the mentioned HPV type. In these cases, degenerated probes were used that is, mix of oligonucleotides each containing alternative nucleotides at the mentioned position. This is the case for probes 39C3d [SEQ ID NO 41], 39C4d [SEQ ID NO 43], 45C1d [SEQ ID NO 57], 45C3d [SEQ ID NO 58], 57A1d [SEQ ID NO 74], 59C3_{—}3d [SEQ ID NO 81], 66B1 [SEQ ID NO 91], 66C3d [SEQ ID NO 93], and 83B1d [SEQ ID NO 126]. Alternatively, equimolecular mixtures of two oligonucleotides comprising exactly the same sequence region but differing on nucleotide composition for certain positions were used as a single probe (mix of oligonucleotide 58B1a [SEQ ID NO 77] and 58B1b [SEQ ID NO 78]; 68C4b [SEQ ID NO 100] and 68C4c [SEQ ID NO 101]; 74A1a [SEQ ID NO 116] and 74A1b [SEQ ID NO 117]; 74B1a [SEQ ID NO 118] and 74B1b [SEQ ID NO 119]; and mix of oligonucleotide 82A2a-AS [SEQ ID NO 122] and 82A2b-AS [SEQ ID NO 123].

All probes disclosed in the present invention have been proved to specifically hybridize to their target sequences under the same hybridization conditions in the ‘array tube’ platform. This fact makes possible the use of these probes for simultaneous identification of 42 different HPV types using this microarray platform. The high number of HPV types identified by the use of the ‘array tube’ developed in the present invention makes this methodology is also considered as a direct detection method, since remaining HPV types are clinically irrelevant.

One of the weak points of diagnostic methods is the appearance of false negatives. In the case of the present method, false negatives can be caused by poor quality DNA samples or by the presence of DNA polymerase inhibitors in the samples to be analyzed. The present invention illustrates the way of eliminating these false negatives via the use of two types of controls.

One control consisting of amplification of the patient's own DNA is preferably used to assure the good quality of DNA sample. Any sequence fragment from human DNA can be used as target for this purpose. A fragment from a single copy gene, such as the CFTR gene, was considered a specially suitable target for positive control of DNA quality in the present invention. Primers CFTR-F4 (SEQ ID NO 134) and CFTR-R5 (SEQ ID NO 135) were designed for amplification of an 892 by fragment from CFTR gene. The use of a single copy versus a multiple copy target and the bigger size of the quality DNA control amplified product compared to the HPV amplified fragment, that is 892 by versus around 450 by respectively, allowed the inclusion of primers for CFTR amplification in the same reaction mixture that the used for the amplification of the L1 region of the HPV genome with minimal competition effects. Therefore, quality DNA control may be simultaneously run in the same reaction tube where the sample is analyzed without affecting to the sensitivity for HPV detection.

A second control may be used as amplification positive control that detects PCR reaction failures due, for example, to the presence of DNA polymerase inhibitors. In a preferred embodiment, amplification positive control consists of a recombinant plasmid that can be amplified using the same primers and the same PCR conditions than those used for amplification of the CFTR gene fragment. Both size and internal sequence to the primers are different between PCR products resulting from amplification of CFTR gene and from amplification of recombinant plasmid. In this way, both types of amplification products can be easily distinguished via gel electrophoresis or via hybridization with specific probes. FIG. 6 shows a schematic representation of recombinant plasmid pPG44 having these characteristics.

Plasmid pPG44 was constructed by molecular cloning techniques. Briefly, a DNA insert consisting of the 1162 by fragment from position 124 to position 1285 of vector pBluescript® II SK+(Stratagene, La Jolla, Calif., USA) flanked by CFTR primers, CFTR-F4 and CFTR-R5, was cloned into pGEM®-T Easy Vector using the commercially available kit from Promega Corporation, Madison, Wis., USA. A purified preparation of obtained recombinant plasmid pPG44 was further characterised by the use of restriction enzymes and by sequence analysis. Plasmid pPG44 was used as positive control of the amplification process in a linearized form.

The presence of a positive control as the mentioned recombinant plasmid in the same PCR amplification mixture where the sample is analyzed prevents the occurrence of false negative results, that is it prevents a negative result from being given even in the presence of the target HPV genome in the sample, because when none of the amplification products are generated it must be assumed that the PCR amplification has not properly worked and a conclusion cannot be drawn as to the presence or absence of the HPV genome in the sample.

Probes for specific detection of the two types of positive controls described, that is DNA quality control and amplification reaction control, are provided in table 2 (SEQ ID NO 136-139 and SEQ ID NO 145-147). Oligonucleotides sequences with no significant homology to any of the amplified products of the present invention are also provided in this table 2 (SEQ ID NO 140-141). When immobilized to the surface of the microarray, biotin modified oligonucleotides SEQ ID NO 140 and SEQ ID NO 141 serve as positive control of the PCR products detection reaction and as positioning reference so that all remaining probes can be located.

TABLE 2

| SEQ | | | |
|---|---|---|---|
| ID NO | Probe name | Control type | Sequence (5′-3′) |
| 136 | CFTR-A1-AS | Sample DNA Quality | TTCTCCACCCACTACGCACCCCCGCCAGCA |
| 137 | CFTR-B3 | Sample DNA Quality | GGGCTCAAGCTCCTAATGCCAAAGACCTACTACTCTG |
| 145 | CFTR-B1-AS | Sample DNA Quality | CAAGCTCCTAATGCCAAAGACCTACTACTC |
| 146 | CFTR-B2 | Sample DNA Quality | GGGCTCAAGCTCCTAATGCCAAAGACCTACTACTC |
| 138 | CIA1-AS | PCR reaction | CTCATTAGGCACCCCAGGCTTTACACTTTAT |
| 139 | CIA2-AS | PCR reaction | TCACTCATTAGGCACCCCAGGCTTTACACTTTATG |
| 147 | CIA3-AS | PCR reaction | GAGTGAGCTGATACCGCTCGCCGCAGCCGAACGAC |
| | | Detection & | |
| 140 | Marker-1 | location | GCAGTATAAGATTATTGATGCCGGAAC |
| | | Detection & | |
| 141 | Marker-2 | location | GTCAAAACCTGGGATAGTAGTTTTACC |

The present invention also relates to an in vitro diagnostic kit for specific detection of HPV types in clinical samples. Preferably, the mentioned kit would include any or all of the following components: amplification mix, including amplification buffer, dNTPs, primers, and control plasmid; wash buffer; detection reagents; array tube including a solid support including HPV type-specific probes; reagents for obtaining and preparing a sample. The particular components will depend on the exact conditions under which the kit is intended to be used, although the skilled person will be able to determine suitable kit components and buffer compositions.

### EXAMPLES

The examples provided below merely illustrate the invention and in no way limit the scope of the accompanying claims.

### Example 1

### Preparation of ‘Array-Tubes’

‘Array tubes’ of the present invention were manufactured at CLONDIAG chip Technologies GmbH (Jena, Germany) as follows. A standard reaction test tube from Eppendorf made of polypropylene and having a nominal receiving volume of 1.5 ml was modified by re-melting, so that, an opened recess for the microarray support with an adhesive edge was modelled into the tube. Microarrays to be inserted into these tubes were produced by using a MicroGrid II Arrayer (BioRobotics, Cambridge, Great Britain). Probes consisting of 5′ end amino-modified oligonucleotides having a sequence from the sequence list were deposited at defined sites on an epoxidized glass surface of a slide (slide size: 75 mm×25 mm) and covalently immobilised. A single microarray included 12×10=120, or 12×11=132 concrete locations at which oligonucleotides could be deposited. These locations have a spacing of 0.2 mm, so that the DNA library included in each microarray covered an area of 2.4 mm×2.4 mm and, in total, more than 100 identical DNA libraries could be produced in this way per slide. Depending on the type of experiment, either one single probe or a mixture of them could be deposited at each one of these locations. Usually, single probes were deposited at each location when specificity and sensitivity experiments for probes selection were carried out. Once the probes have been validated, mixtures of probes capable of hybridizing in separate regions of the amplified product of a specific HPV type could be deposited in the same location when identification of HPV genotypes assays were performed. FIGS. 1 to 5 show different arrangements of probes within microarrays used for this invention. Two or three replicates for each probe or mixture of probes were included in each microarray.

Besides specific probes for HPV genotyping and for detection of amplification control and adequacy of DNA control, microarrays included reference markers at several locations consisting of 5′ end biotin modified oligonucleotides (Marker-1 [SEQ ID NO 140] and Marker-2 [SEQ ID NO 141]) with no significant homology for any of the amplified sequences from this invention. These reference markers served both for verifying proper performance of the detection reaction and for optical orientation of the image by the reader so all remaining probes can be located and the data analyzed.

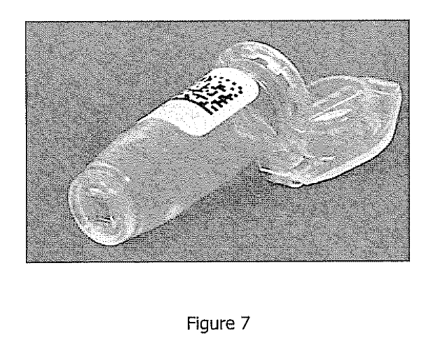

All oligonucleotides were deposited on the slide from a 1× QMT Spotting Solution I (Quantifoil Micro Tools GmbH, Jena, Germany). Total concentration of oligonucleotides in each spotting solution ranged from 2.5 μM for reference markers to 20 μM for specific probes. Oligonucleotides were then covalently linked to the epoxide groups on the glass surface by baking at 60° C. for 30 minutes followed by a multi-step washing process. Dried slides were cut into 3.15 mm×3.15 mm glass pieces which, strictly speaking, are what we name microarrays. In the final step for ‘array tubes’ manufacturing, these microarrays were then inserted into the aforementioned modified Eppendorf tubes and glued to the adhesive edge. FIG. 7 shows a photograph of an ‘array tube’ produced as specified in the present example.

### Example 2

### Preparation of DNA Samples

### 2.1. HPV DNA Standards

HPV DNAs used to assess the specificity and sensitivity of type-specific probes were either recombinant plasmids containing the amplified L1 region (HPV types 6, 11, 13, 16, 18, 26, 31, 33, 35, 39, 40, 42, 44, 45, 51, 52, 53, 54, 56, 58, 61, 62, 66, 68, 70, 71, 72, 73, 81, 82, 83, 84, 85 and 89) or DNAs extracted from clinical samples which amplified L1 region was further characterized by DNA sequencing. Recombinant plasmids were constructed by molecular cloning techniques. Briefly, amplified L1 region from each HPV type was cloned into pGEM®-T Easy Vector using the commercially available kit from Promega Corporation, Madison, Wis., USA. A purified preparation obtained from each recombinant plasmid was further characterised by sequence analysis. From 1 to 10 pg of plasmid DNA were used in assessment of specificity experiments.

DNA from the K562 cell line (Catalogue No. DD2011, Promega Corporation, Madison, Wis., USA) served to assess the specificity and sensitivity of CFTR specific probes.

### 2.2. Clinical Samples

For the purpose of detecting HPV, it is first of all necessary to separate DNA from remaining biological material. Preparation of DNA procedures vary according to sample source. Specific examples are provided for preparation of DNA from samples from a variety of sources:

A. Swabs: samples were taken with a clean, dry, cotton swab. Cells from clinical swabs were recovered by addition of 1.5 ml of saline directly to the container with the sample and vigorous vortexing. Sample material was transferred to a 1.5 ml Eppendorf tube and pelleted by centrifugation. The supernatant was discarded and the precipitated cells were suspended in 100 μl of lysis buffer containing 10 mM Tris-HCl (pH 9.0 at 25° C.), 50 mM KCl, 0.15 mM MgCl₂, 0.1% Triton® X-100, 0.5% Tween 20, and 0.25 mg/ml Proteinase K. This mixture was incubated at 56° C. for about 2 hours, and the proteinase K was heat-inactivated by incubating the mixture at 100° C. for 10 minutes. Detritus was pelleted by centrifugation and supernatant was transferred to a clean and sterile tube. An Aliquot of 5 μl was subsequently used in the PCR reaction.

B. Cell suspensions: this type of sample refers to that used in cervicovaginal liquid based cytology tests. Cervical specimens were taken with a brush or spatula and resuspended in PreservCyt solution (Cytyc Corp., Marlborough, Mass., USA). An aliquot of 1 ml was centrifuged and the pellet was resuspended in 1 ml of saline. After a new centrifugation step, pellet was resuspended in 100 μl of lysis buffer as that used with the swabs samples in paragraph A and protocol was continued in the same way as in that section.

C. Formalin fixed and paraffin-embedded biopsies: several tissue sections of 5 μm in width were used in the present method, typically 2-5 sections, depending on the surface area from the biopsy. Sections were placed in a 1.5 ml sterile tube and 100 μl of lysis buffer as that used with the swabs samples in paragraph A were added. Protocol was continued in the same way as in that section, except that incubation with Proteinase K was carried out for 3 hours.

Alternatively, a commercial kit (NucleoSpin®) Tissue kit Catalogue No. 635966 from BD Biosciences Clontech, Palo Alto, Calif., USA) designed for DNA isolation from samples from a variety of sources was used to process swabs, cell suspensions or formalin fixed and paraffin-embedded biopsies samples. In this case, the beginning of the DNA isolation protocol was as specified in sections A, B and C. Instead of 100 μl of lysis buffer, 180 μl of Buffer T1 was added to the sample. Protocol was continued following manufacturer specifications for isolation of genomic DNA from cells and tissue.

Whatever it was the type of clinical sample or the DNA preparation method, negative controls were run in parallel with each batch of samples. These negative controls constituted of 1 ml of saline were processed in the same way as in section A.

### Example 3

### PCR Amplification

PCR amplification using consensus primers MY11 and MY09 (Manos et al., Molecular Diagnostics of Human Cancer; Furth M, Greaves M F, eds.; Cold Spring Harbor Press. 1989, vol. 7: 209-214) was performed. A third primer, HMB01, that is often used in combination with MY09 and MY11 to amplify HPV type 51 which is not amplified efficiently with MY09 and MY11 alone (Hildesheim et al., J Infect Dis. 1994, 169: 235-240), was also included in the PCR reaction. Briefly, PCR amplification was carried out in a 50 μl final volume reaction containing 10 mM Tris-HCl pH 8.3, 50 mM KCl, 1 mM MgCl₂, 0.3 μl each primer MY09 and MY11 (SEQ ID NO 142 and 143), 0.03 μM primer HMB01 (SEQ ID NO 144), 200 μM of each dNTP, 4 units of AmpliTaq Gold DNA polymerase (Applied Biosystems, Foster City, Calif., USA), and 5 μl of each HPV DNA standard from Example 2.1. or clinical sample DNA from Example 2.2. To test the suitability of sample DNA, 0.08 μM each primer CFTR-F4 and CFTR-R5 (SEQ ID NO 134 and 135) was also added to the reaction mixture. Additionally, to check amplification process and eliminate false negatives results due to reaction failure 20 fg of internal control pPG44 was included in the same reaction tube in which the samples were analysed. All forward primers used in the PCR reaction (MY11 [Seq ID NO 143] and CFTR-F4 [Seq ID NO 134]) were biotin modified at the 5′ end so that any amplified DNA could be subsequently detected.

Negative controls constituted of 5 μl of blank samples from Example 2.2. or 5 μl of deionised water were processed in parallel with the samples DNA. The use of these kinds of negative controls serves to check that contamination does not occur at any point in sample handling or in PCR reaction setting up and all positive results represent true presence of DNA in the sample.

PCR reactions were run in a Mastercycler thermocycler (Eppendorf, Hamburg, Germany) programmed with the following cycling profile: one initial denaturing cycle at 95° C. for 9 minutes, 45 cycles of 30 seconds at 94° C., 60 seconds at 55° C. and 90 seconds at 72° C., and one final extension cycle at 72° C. for 8 minutes. After amplification, 5 μl of each reaction were used for subsequent detection with specific probes.

### Example 4

### Simultaneous Identification of HPV Genotypes Using ‘Array Tubes’

‘Array tubes’ were pre-washed just before its use by addition of 300 μl of 0.5× PBS-Tween 20 buffer to each tube and inverting them several times. All liquid from inside each tube was removed using a Pasteur pipette connected with a vacuum system.

Amplification reactions from Example 3 were denatured by heating them to 95° C. for 10 minutes and, immediately after, cooling them down for 5 minutes on ice. Five microlitres of denatured amplification reaction were applied to the ‘array tube’ prepared in Example 1 together with 100 μl of hybridization solution (250 mM sodium phosphate buffer, pH 7.2; SSC 1×; 0.2% Triton® X-100; 1 mM EDTA, pH 8.0). Hybridization reaction was carried out in a Thermomixer comfort (Eppendorf, Hamburg, Germany) by incubating the ‘array tubes’ at 55° C. for one hour with shaking at 550 rpm. After incubation period, hybridization reaction was removed using a Pasteur pipette connected with a vacuum system and a washing step with 300 μl of 0.5× PBS-Tween 20 buffer was carried out.

Hybridized DNA was detected by incubation in 100 μl of a 0.075 μg/ml Poly-HRP Streptavidin (Pierce Biotechnology Inc., Rockford, Ill., USA) solution at 30° C. for 15 minutes with shaking at 550 rpm. Then, all liquid from the ‘array tube’ was quickly removed and two washing steps as that aforementioned were carried out. Colour developing reaction was performed in 100 μl of True Blue™ Peroxidase Substrate (KPL, Gaithersburg, Md., USA), which consists of a buffered solution containing 3,3′,5,5′-tetramethylbenzidine (TMB) and H₂O₂, by incubation at 25° C. for 10 minutes. The coloured precipitates so produced cause changes in the optical transmission at concrete locations of the microarray that can be read using an ATRO1 or an ATS reader manufactured by CLONDIAG chip technologies GmbH (Jena, Germany). Optionally, ATS reader may have specific software installed for automatic processing of the sample analysis result obtained with the ‘array tube’ developed in the present invention.

1. An assay for detecting and typing human papillomavirus (HPV) in a sample, the assay comprising:
performing a nucleic acid amplification reaction on a sample, the amplification reaction being intended to amplify an HPV target sequence in a non-type specific manner;
obtaining single stranded oligonucleotides from any amplification products;
allowing single stranded oligonucleotides to hybridise where possible with the a plurality of HPV type-specific probes provided on a solid support, the support being located within a reaction vessel suitable for containing the sample; and
detecting hybridised oligonucleotides.

2. The assay of claim 1 wherein said HPV type-specific probes comprise DNA.

3. The assay of claim 1 or 2 wherein the nucleic acid amplification step is carried out on the sample within the reaction vessel in contact with the HPV type-specific probes on the solid support.

4. The assay of claim 1 or 2 wherein the nucleic acid amplification step is carried out on the sample prior to introduction of the amplified sample to the reaction vessel to contact the HPV type-specific probes on the solid support.

5. The assay of any preceding claim wherein the probes are selected to specifically bind to the HPV target sequence under the same hybridisation conditions for all probes.

6. The assay of any preceding claim wherein probes specific for at least 20 HPV types are used.

7. The assay of any preceding claim wherein probes specific for at least 20 of HPV types 6, 11, 16, 18, 26, 30, 31, 32, 33, 34/64, 35, 39, 40, 42, 43, 44, 45, 51, 52, 53, 54, 56, 57, 58, 59, 61, 62, 66, 67, 68, 69, 70, 71, 72, 73, 74, 81, 82, 83, 84, 85 and 89 are used.

8. The assay of any preceding claim wherein the probes are 20 to 40 nt in length.

9. The assay of any preceding claim wherein the probes are 25 to 35 nt.

10. The assay of any preceding claim wherein the probes are 28 to 32 nt.

11. The assay of any preceding claim wherein the probes are around 30 nt.

12. The assay of any preceding claim wherein the probes are specific to the L1 region of HPV.

13. The assay of any preceding claim wherein each probe differs from probes specific to another HPV type in at least 2 nt.

14. The assay of any preceding claim wherein each probe differs from probes specific to another HPV type in at least 3 nt.

15. The assay of any preceding claim wherein one or more of the probes are selected from the group comprising SEQ ID NO 1 to SEQ ID NO 133.

16. The assay of any preceding claim wherein all of the probes are selected from the group comprising SEQ ID NO 1 to SEQ ID NO 133.

17. The assay of any preceding claim wherein a plurality of the probes are selected from one or more of the following groups of SEQ IDs : 1 or 2; 3 or 4; 5 to 9; 10 to 13; 14 to 18; 19, 20, or 21; 22 to 25; 26 or 27; 28 to 31; 32 or 33; 34 to 37; 38 to 43; 44 or 45; 46 to 50; 51 or 52; 53 or 54; 55 to 59; 60 to 64; 65 or 66; 67 or 68; 69, 70 or 71; 72 or 73; 74 or 75; 76, 77, or 78; 79 to 83; 84, 85, or 86; 87, 88, or 89; 90 to 94; 95, 96 or 97; 98 to 102; 103 or 104; 105 or 106; 107 or 108; 109 or 110; 111 to 115; 116 to 119; 120 or 121; 122, 123, or 124; 125 or 126; 127 or 128; 129 or 130; 131, 132 or 133.

18. The assay of claim 17 wherein a probe is selected from each of the said groups.

19. The assay of claim 17 wherein each probe is selected from the said groups, and at least one probe is selected from each of the said groups.

20. The assay of claim 17 wherein two or more probes are selected from each of the said groups.

21. The assay of any preceding claim wherein the probes are selected from the following SEQ IDs : 2, 4, 7, 8, 9, 12, 13, 16, 17, 18, 19, 20, 21, 24, 25, 26, 27, 30, 31, 32, 33, 36, 37, 40, 41, 42, 43, 45, 48, 49, 50, 51, 52, 53, 54, 57, 58, 59, 61, 62, 63, 64, 66, 67, 68, 70, 71, 73, 74, 75, 76, 81, 82, 83, 84, 85, 86, 87, 88, 89, 91, 92, 93, 94, 95, 96, 97, 100, 101, 102, 103, 104, 105, 106, 107, 108, 109, 110, 112, 114, 115, 116, 117, 118, 119, 120, 121, 124, 126, 128, 129, 130, 131, 132, 133.

22. The assay of any preceding claim wherein a plurality of probes are specific for the same HPV type.

23. The assay of any preceding claim wherein a plurality of probes are specific for each HPV type to be detected.

24. The assay of any of claim 22 or 23 wherein each of said plurality of probes is immobilised to the same region of the solid support.

25. The assay of any of claims 22 to 23 wherein each of said plurality of probes is immobilised to a distinct region of the solid support.

26. The assay of any of claims 23 to 25 wherein each probe specific for the same HPV type detects a different portion of the HPV target sequence.

27. The assay of any preceding claim wherein at least one probe is present on the solid support in at least two distinct locations.

28. The assay of any preceding claim wherein all probes are present on the solid support in at least two distinct locations.

29. The assay of any preceding claim further comprising detecting one or more control sequences.

30. The assay of claim 29 wherein the control sequence comprises a probe immobilised to the solid support which does not hybridise to the target sequence from any HPV type.

31. The assay of claim 29 wherein the control sequence comprises a human genomic target sequence.

32. The assay of claim 31 wherein the human target sequence comprises at least a portion of the CFTR gene.

33. The assay of any preceding claim further comprising amplifying a known control sequence, and detecting the amplification product.

34. The assay of any preceding claim comprising combining an amplification reaction mix with the sample to perform the amplification reaction.

35. The assay of any preceding claim, wherein the amplification reaction is PCR.

36. The assay of any preceding claim, wherein single stranded oligonucleotides are obtained by denaturing any double stranded oligonucleotides present.

37. The assay of claim 36, wherein said denaturing step is carried out on a sample contained within the reaction vessel.

38. The assay of any preceding claim, wherein single stranded oligonucleotides are allowed to hybridise under stringent conditions.

39. An assay for detecting and typing human papillomavirus (HPV) in a sample, the assay comprising:
performing a nucleic acid amplification reaction on a sample in a reaction vessel comprising a solid support having a plurality of HPV type-specific probes immobilised thereon, the amplification reaction being intended to amplify an HPV target sequence in a non-type specific manner;
obtaining single stranded oligonucleotides from any amplification products;
allowing single stranded oligonucleotides to hybridise where possible with the HPV type-specific probes; and
detecting hybridised oligonucleotides;
wherein the amplification reaction takes place in the sample in contact with the solid support.

40. A reaction vessel for performing an assay for detecting and typing HPV in a sample, the vessel comprising a solid support having a plurality of HPV type-specific probes immobilised thereon, and being suitable for containing a sample in contact with the solid support.

41. The vessel of claim 40 wherein the vessel is suitable for performing a nucleic acid amplification reaction on a sample in contact with the solid support.

42. The vessel of claim 40 or 41 wherein the probes are selected to specifically bind HPV target sequences under the same hybridisation conditions for all probes.

43. The vessel of any of claims 40 to 42 wherein the probes are selected to specifically bind HPV target sequences in a sample comprising a reaction mix suitable for carrying out a nucleic acid amplification reaction.

44. The vessel of any of claims 40 to 43 wherein said HPV type-specific probes comprise DNA.

45. The vessel of any of claims 40 to 44 comprising probes specific for at least 20 HPV types.

46. The vessel of any of claims 40 to 44 comprising probes specific for at least 20 of HPV types 6, 11, 16, 18, 26, 30, 31, 32, 33, 34/64, 35, 39, 40, 42, 43, 44, 45, 51, 52, 53, 54, 56, 57, 58, 59, 61, 62, 66, 67, 68, 69, 70, 71, 72, 73, 74, 81, 82, 83, 84, 85 and 89.

47. The vessel of any of claims 40 to 46 wherein the probes are 20 to 40 nt in length.

48. The vessel of any of claims 40 to 47 wherein the probes are 25 to 35 nt.

49. The vessel of any of claims 40 to 48 wherein the probes are 28 to 32 nt.

50. The vessel of any of claims 40 to 49 wherein the probes are around 30 nt.

51. The vessel of any of claims 40 to 50 wherein the probes are specific to the L1 region of HPV.

52. The vessel of any of claims 40 to 51 wherein each probe for a specific HPV type differs from probes specific to another HPV type in at least 2 nt.

53. The vessel of any of claims 40 to 52 wherein each probe for a specific HPV type differs from probes specific to another HPV type in at least 3 nt.

54. The vessel of any of claims 40 to 53 wherein one or more of the probes are selected from the group comprising SEQ ID NO 1 to SEQ ID NO 133.

55. The vessel of any of claims 40 to 54 wherein all of the probes are selected from the group comprising SEQ ID NO 1 to SEQ ID NO 133.

56. The vessel of any of claims 40 to 55 wherein a plurality of the probes are selected from one or more of the following groups of SEQ IDs : 1 or 2; 3 or 4; 5 to 9; 10 to 13; 14 to 18; 19, 20, or 21; 22 to 25; 26 or 27; 28 to 31; 32 or 33; 34 to 37; 38 to 43; 44 or 45; 46 to 50; 51 or 52; 53 or 54; 55 to 59; 60 to 64; 65 or 66; 67 or 68; 69, 70 or 71; 72 or 73; 74 or 75; 76, 77, or 78; 79 to 83; 84, 85, or 86; 87, 88, or 89; 90 to 94; 95, 96 or 97; 98 to 102; 103 or 104; 105 or 106; 107 or 108; 109 or 110; 111 to 115; 116 to 119; 120 or 121; 122, 123, or 124; 125 or 126; 127 or 128; 129 or 130; 131, 132 or 133.

57. The vessel of claim 56 wherein a probe is selected from each of the said groups.

58. The vessel of claim 56 wherein each probe is selected from the said groups, and at least one probe is selected from each of the said groups.

59. The vessel of claim 56 wherein two or more probes are selected from each of the said groups.

60. The vessel of any of claims 40 to 59 wherein the probes are selected from the following SEQ IDs : 2, 4, 7, 8, 9, 12, 13, 16, 17, 18, 19, 20, 21, 24, 25, 26, 27, 30, 31, 32, 33, 36, 37, 40, 41, 42, 43, 45, 48, 49, 50, 51, 52, 53, 54, 57, 58, 59, 61, 62, 63, 64, 66, 67, 68, 70, 71, 73, 74, 75, 76, 81, 82, 83, 84, 85, 86, 87, 88, 89, 91, 92, 93, 94, 95, 96, 97, 100, 101, 102, 103, 104, 105, 106, 107, 108, 109, 110, 112, 114, 115, 116, 117, 118, 119, 120, 121, 124, 126, 128, 129, 130, 131, 132, 133.

61. The vessel of any of claims 40 to 60 wherein a plurality of probes are specific for the same HPV type.

62. The vessel of any of claims 40 to 61 wherein a plurality of probes are specific for each HPV type to be detected.

63. The vessel of any of claim 61 or 62 wherein each of said plurality of probes is immobilised to the same region of the solid support.

64. The vessel of any of claim 61 or 62 wherein each of said plurality of probes is immobilised to a distinct region of the solid support.

65. The vessel of any of claims 62 to 64 wherein each probe specific for the same HPV type detects a different portion of the HPV target sequence.

66. The vessel of any of claims 40 to 65 wherein at least one probe species is present on the solid support in at least two distinct locations.

67. The vessel of any of claims 40 to 66 wherein all probe species are present on the solid support in at least two distinct locations.

68. The vessel of any of claims 40 to 67 further comprising one or more control sequences on the solid support.

69. The vessel of claim 68 wherein the control sequence comprises a probe immobilised to the solid support which does not hybridise to the target sequence from any HPV type.

70. The vessel of claim 68 wherein the control sequence comprises a human genomic target sequence.

71. The vessel of claim 70 wherein the human target sequence comprises at least a portion of the CFR gene.

72. A kit for the detection and typing of HPV comprising the reaction vessel of any of claims 40 to 71, in combination with one or more of the following:
i) reagents for DNA extraction and/or purification;
ii) a nucleic acid amplification mix;
iii) reagents for use in visualising hybridisation of nucleic acids to the probes of the reaction vessel,

73. The kit of claim 72 wherein the amplification mix is provided in a separate reaction vessel from the reaction vessel comprising the solid support with HPV type-specific probes.

74. The kit of claim 72 wherein the amplification mix is provided in the reaction vessel comprising the solid support with HPV type-specific probes.

75. The kit of any of claims 72 to 74 wherein the amplification mix comprises labelled dNTPs.

76. The kit of any of claims 72 to 75 wherein the amplification mix comprises HPV consensus primers which hybridise to portions of the HPV target sequence.

77. The kit of claim 76 wherein the HPV consensus primers comprise MY09 and MY11; and optionally HMBO1.

78. The kit of any of claims 72 to 77 wherein the amplification mix comprises primers for amplifying a human target sequence.

79. The kit of claim 78 wherein the human target sequence is of a different length to the HPV target sequence.

80. The kit of claim 78 or 79 wherein the human target sequence is at least a portion of the CFM gene.

81. The kit of claim 80 wherein the primers comprise at least one of CFTR-F4 (SEQ ID NO 134) and CFTR-R5 (SEQ ID NO 135).

82. The kit of any of claims 76 to 81 wherein the primers are labelled primers.

83. The kit of any of claims 72 to 82 comprising a control amplification target sequence.

84. The kit of claim 83 wherein the control amplification target sequence includes sequences corresponding to flanking portions of the human target sequence, such that amplification of both target sequences will occur using the same primers.

85. A probe for detecting and typing HPV, the probe being selected from SEQ ID NO 1 to 133.

86. The probe of claim 85, selected from the following SEQ IDs : 2, 4, 7, 8, 9, 12, 13, 16, 17, 18, 19, 20, 21, 24, 25, 26, 27, 30, 31, 32, 33, 36, 37, 40, 41, 42, 43, 45, 48, 49, 50, 51, 52, 53, 54, 57, 58, 59, 61, 62, 63, 64, 66, 67, 68, 70, 71, 73, 74, 75, 76, 81, 82, 83, 84, 85, 86, 87, 88, 89, 91, 92, 93, 94, 95, 96, 97, 100, 101, 102, 103, 104, 105, 106, 107, 108, 109, 110, 112, 114, 115, 116, 117, 118, 119, 120, 121, 124, 126, 128, 129, 130, 131, 132, 133.

87. A primer for use in amplifying CFTR, the primer selected from CFTR-F4 (SEQ ID NO 134) and CFTR-R5 (SEQ ID NO 135).
